(19) United States
(12) Patent Application Publication
(10) Pub. No.: US 2018/0325409 A1
(43) Pub. Date: Nov. 15, 2018

(54) Data-Integrated Interface and Methods of Reviewing Electromyography and Audio Data

(71) Applicant: BRAIN SENTINEL, INC., San Antonio, TX (US)

(72) Inventor: Michael R. Girouard, Shavano Park, TX (US)

(73) Assignee: BRAIN SENTINEL, INC., San Antonio, TX (US)

(21) Appl. No.: 15/103,562

(22) PCT Filed: Dec. 12, 2014
(86) PCT No.: PCT/US14/70118

§ 371 Date: Jul. 20, 2016

Related U.S. Application Data
(60) Provisional application No. 62/050,054, filed on Sep. 12, 2014.
(60) Provisional application No. 62/032,147, filed on Aug. 1, 2014.
(60) Provisional application No. 62/001,302, filed on May 21, 2014.
(60) Provisional application No. 61/979,225, filed on Apr. 14, 2014.
(60) Provisional application No. 61/969,660, filed on Mar. 24, 2014.
(60) Provisional application No. 61/915,236, filed on Dec. 12, 2013.

(51) Int. Cl. A61B 5/0492; A61B 5/00; G16H 80/00; G16H 10/60

(52) U.S. Cl. CPC A61B 5/0492; A61B 5/4094; G16H 80/00; A61B 2505/07; A61B 5/0004; A61B 5/0022; A61B 2505/01; G16H 10/60

(57) ABSTRACT

A method of reviewing medical data for a patient susceptible to seizure activity may include monitoring the patient using electromyography electrodes and acoustic sensors. Data may be collected and organized for review and analysis by processing of the data to facilitate graphing of electromyography data and enabling a user to play an audio recording of detected sounds.

### CROSS REFERENCE TO RELATED APPLICATIONS

This application claims priority to U.S. Provisional Patent Application No. 61/915,236 filed Dec. 12, 2013, U.S. Provisional Patent Application No. 62/050,054 filed Sep. 12, 2014, U.S. Provisional Patent Application No. 62/032,147 filed Aug. 1, 2014, U.S. Provisional Patent Application No. 62/001,302 filed May 21, 2014, U.S. Provisional Patent Application No. 61/979,225 filed Apr. 14, 2014, and U.S. Provisional Patent Application No. 61/969,660 filed Mar. 24, 2014. This application is also a continuation-in-part of PCT Patent Application No. PCT/US14/54837, which claims priority to U.S. Provisional Patent Application No. 61/875,429 filed Sep. 9, 2013. This application is also a continuation-in-part of U.S. patent application Ser. No. 13/275,309 filed Oct. 17, 2011, which claims priority to U.S. Provisional Patent Application Ser. No. 61/393,747 filed Oct. 15, 2010 and U.S. patent application Ser. No. 13/542,596 filed Jul. 7, 2012, which claims priority to U.S. Provisional Patent Application Ser. No. 61/504,582 filed Jul. 5, 2011. The disclosures of all of the above are herein fully incorporated by reference.

### BACKGROUND

A seizure may be characterized as abnormal or excessive synchronous activity in the brain. At the beginning of a seizure, neurons in the brain may begin to fire at a particular location. As the seizure progresses, this firing of neurons may spread across the brain, and in some cases, many areas of the brain may become engulfed in this activity. Seizure activity in the brain may cause the brain to send electrical signals through the peripheral nervous system to different muscles the activation of which may initiate a redistribution of ions within muscle fibers. In electromyography (EMG), an electrode may be placed on or near the skin and configured to measure changes in electrical potential resulting from ion flow during this muscle activation.

EMG detection may be particularly amenable for use in apparatuses that may be minimally intrusive, minimally interfere with daily activities and which may be comfortably used while sleeping. Therefore, methods of monitoring the seizure activity of patients, including methods for monitoring in ambulatory or home settings, may benefit from the use of EMG detection. For some patients, a seizure event may also be presented as an audible scream or vocalization which may typically occur at the start of a seizure. Like EMG detection, audio detection of seizures may be particularly amenable to methods of patient monitoring that may be minimally intrusive, and monitoring of seizure activity using one or more acoustic sensors in combination with EMG may be used in detection methods.

### SUMMARY

In some embodiments, a method of reviewing data collected for a patient susceptible to seizure activity may include monitoring a patient using an EMG electrode and an acoustic sensor; determining whether a signal received by the EMG electrode and/or the acoustic sensor exceeds a threshold value; if said threshold value is achieved sending EMG and audio data to a remote server and storing the data on the remove server: installing on a remote device instructions for accessing and processing the stored EMG data and the stored audio data on said remote server; wherein the processing of said EMG data and said audio data includes graphing the EMG data and enabling a user to play an audio recording of sounds detected by said acoustic sensor; wherein a marker is shown on the graphed EMG data synchronizing the played audio recording with a specific segment of the graphed EMG data.

### BRIEF DESCRIPTION OF THE DRAWINGS

FIG. 1 illustrates one embodiment of a seizure detection system.

FIG. 2 illustrates a display for selecting a seizure related event for a patient.

FIG. 3 illustrates a display of EMG data for selected data.

FIG. 4 illustrates another display of EMG data for another portion of selected data.

FIG. 5 illustrates yet another display of EMG data for another portion of selected data.

FIG. 6 illustrates a split-screen view showing two spectral regions of EMG data.

### DETAILED DESCRIPTION

The following terms as used herein should be understood to have the indicated meanings.

When an item is introduced by “a” or “an,”it should be understood to mean one or more of that item.

“Comprises” means includes but is not limited to.

“Comprising” means including but not limited to.

“Computer” means any programmable machine capable of executing machine-readable instructions. A computer may include but is not limited to a general purpose computer, microprocessor, computer server, digital signal processor, or a combination thereof. A computer may comprise one or more processors, which may comprise part of a single machine or multiple machines.

“Computer program” means a list of instructions that may be executed by a computer to cause the computer to operate in a desired manner.

“Computer readable medium” means an article of manufacture having a capacity for storing one or more computer programs, one or more pieces of data, or a combination thereof. A computer readable medium may include but is not limited to a computer memory, hard disk, memory stick, magnetic tape, floppy disk, optical disk (such as a CD or DVD), zip drive, or combination thereof.

The term “having” means including but not limited to.

The apparatuses and methods described herein may be used to detect seizures and timely alert caregivers of seizure-related events using one or more sensors. Sensors may include electrodes attached to a patient or patient's clothing and may be configured for measurement of muscle electrical activity using electromyography (EMG). An acoustic sensor may further be used to monitor a patient and detect characteristic sounds associated with a seizure. Detection of seizures using EMG electrodes or audio sensors is further described in Applicant's U.S. patent application Ser. Nos. 13/275,309 13/542,596 and Applicant's U.S. Provisional Patent Application Nos. 61/875,429, 61/894,793, and 61/875,429 the disclosures of each of which are herein fully incorporated by reference. For example, audio data may be collected, analyzed in real-time, and used in making a decision about whether to alert a caregiver that a patient may be experiencing a seizure. Collected audio data may also be analyzed at times after a period of monitoring and may be used to verify whether a seizure or seizure related event has occurred. In some embodiments, review of audio and EMG data may facilitate classification of seizure and seizure related events and may be used to update settings in a detection algorithm appropriate for use with a particular patient or patient demographic. As described herein, an interface suitable for review of synchronized EMG and audio data is described.

Audio data may include information suitable to replay sounds and may, in some embodiments, farther include characteristic values determined or calculated from signal collected by one or more acoustic sensors. For example, audio data may, by way of nonlimiting example, include audio signal intensity or amplitude, amplitude at a given frequency (or over a certain frequency range), rate of change of amplitude, spectral slope, periodicity, other data, or combinations of audio characteristics thereof. In some embodiments, audio data may be used to calculate one or more input values for use in a seizure detection algorithm, and in some embodiments, the algorithm may operate without operator review. Characteristics of data calculated from an acoustic sensor may, in some embodiments, be displayed along with EMG data. For example, a computer configured to display EMG data may include a tab or window that allows access to one or more graphs displaying amplitude or other characteristics of data that may be derived from an acoustic sensor. Audio data suitable to replay sounds may, furthermore, in some embodiments, be recorded and sent to a remote server for access and review by physicians or other authorized persons. For example, audio data may be sent to a remote server and accessed for analysis on an authorized person's computer, tablet, mobile phone or other device capable of receiving and/or playing an audio recording.

In some embodiments, detection of a seizure or possible seizure related event may trigger automatic transmission of EMG and audio data to a remote monitoring facility. For example, if an alarm is triggered data proceeding and after the event may be sent for review. In some embodiments, data may be stored in a circular buffer for at least some period of time. If a seizure or possible seizure related event is detected data may be sent to a remote device or server for further analysis or review. I no seizure or possible seizure event is detected the information may, in some embodiments, be deleted such as to conserve computer memory. In some embodiments, data may be decimated or compressed to reduce the size of the file, but not reduced so much as to lose visible or audio quality of data. Reduction of the file may, for example, make it more responsive when manipulating the data from a local computer with internet service. A caregiver viewing the data on a local computer may then select to view/listen to any portion of the transmitted data. In one embodiment, a five minute interval on either side of an expected event (e.g., 10 minutes of data) may be sent and/or uploaded for review. A caregiver viewing the data on a local computer may select to view/listen to the entire ten minutes or select on a series of buttons labeled 1-10 to view/listen at a particular 1 minute segment. An apparatus for display of data may be configured such that a selected portion of EMG data may scroll across a screen at a rate such that associated audio data (e.g., data collected at the same time as the EMG data) is simultaneously heard. Examples of some embodiments of data displays are further shown in Examples 1-4 included herein.

In some embodiments, upon selection of a portion of EMG data and selection to play recorded sound, a slider or bar may appear on the viewing screen and the slider may move across the EMG data or along a line beneath the data at a rate that is synchronized with a playing of recorded sound. Therefore, a viewer may be able to simultaneously view the EMG data and hear sound that was recorded at the same time as a designated portion of the collected EMG data. The audio slider may, for example, appear as a bar or marker along the x-axis of a graph showing EMG data on the y-axis and time along the x-axis or along a slider or bar that may be displayed underneath the time axis of EMG data. In some embodiments, a time marker may be displayed as a dashed line, line of other color, bar or displayed using one or more other characteristic features suitable to readily differentiate the marker from associated EMG data. In some embodiments, an audio slider may remain at a fixed location on the viewing screen, and EMG data may move across the screen as a sound recording is played. In other embodiments, EMG data may be displayed on a screen and the audio slider may move across the screen, e.g., from left to right along the screen, as the sound recording is played. A user may further, in some embodiments, be able to select a preference wherein either the audio slider or EMG data move across the screen during event review.

In some embodiments, a method of monitoring a patient for seizure activity may include collecting EMG signal and isolation of spectral data form one or more frequency bands. In some embodiments, a device may be configured to graphically display a total EMG signal, e.g., an integrated signal over all collected frequencies, or a signal from one or more frequency bands. For example, a device may be configured with tabs to toggle a display between different frequency bands or the total EMG signal. In some embodiments, one or more graphs showing EMG data from different frequency bands may be shown in a split-screen configuration on a device.

In some embodiments, graphed EMG data or a played audio recording may further include a designation or marking of when a threshold level of EMG or acoustic data was met. For example, a line or marker on the EMG graph may show one or more positions or regions where a threshold detection of EMG amplitude was reached. For example, a portion of EMG data that exceeds a threshold level of activity may be marked by a shaded region or by one or more vertical lines showing, for example, a rising and falling edge of a detected data peak. Alternatively, in some embodiments, a user may choose to display a threshold level on a graph as a horizontal line designating, e.g., an EMG amplitude that for the particular patient exceeds a threshold.

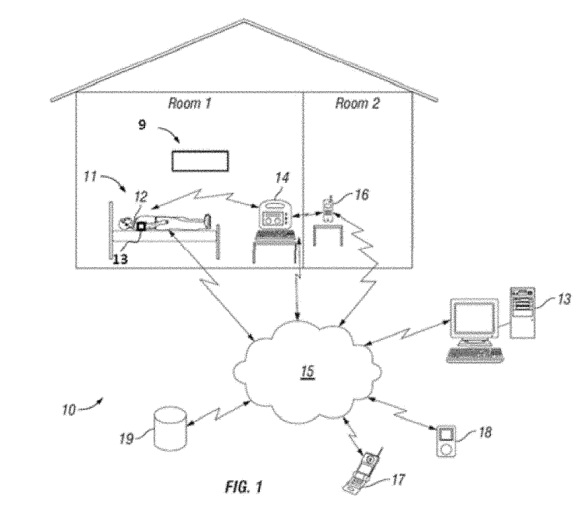

A variety of systems may be suitably used for collecting EMG, audio, and other patient-related data, prioritizing data for storage, organizing such data for system optimization, display of data in a manner to facilitate rapid interpretation of the data and/or initiating an alarm in response to a suspected seizure. FIG. 1 illustrates an exemplary embodiment of such a system. In the embodiment of FIG. 1, a seizure detection system 10 may include a video camera 9, a detection unit 12, an acoustic sensor 13, a base station 14, and an alert transceiver 16. The detection unit may comprise one or more EMG electrodes capable of detecting electrical signals from muscles at or near the skin surface of a patient, and delivering those electrical EMG signals to a processor for processing. The base station may comprise a computer capable of receiving and processing EMG signals from the detection unit and/or acoustic data from an acoustic sensor, determining from the processed EMG and/or acoustic signals whether a seizure may have occurred, and sending an alert to a caregiver. An alert transceiver may be carried by, or placed near, a caregiver to receive and relay alerts transmitted by the base station. Other components that may be included in the system 10, including for example, an alert transceiver 16, wireless device(s) 17, 18, storage database 19, and one or more environmental transceivers are described in greater detail in Applicant's U.S. patent application Ser. Nos. 13/275,309 and 13/542,596.

Devices 17, 18 that may access data may include, for example, an authorized person's computer, tablet, mobile phone or other device capable of receiving, displaying and/or playing data. In some embodiments, processing of data on a device may include installing a computer program, e.g., installing the program on one or more elements of computer readable media. A computer readable medium may include but is not limited to a computer memory, hard disk, memory stick, magnetic tape, floppy disk, optical disk (such as a CD or DVD), zip drive, or combination thereof.

An apparatus for detecting seizures may be man-portable, and may include a detection unit that may be attached to the body, such as by use of an elastic arm band. In some embodiments, EMG electrodes may be implanted within the tissue of a patient near a muscle that may be activated during a seizure. Implanted devices may be particularly amenable for some patients where EMG signals may typically be weak such as, for example, patients with significant adipose tissue.

The detection unit may be battery powered, and may wirelessly communicate with the base station or may be configured to send information directly to a remote database or server. The detection unit may include sufficient data storage, processing and transmission capability to receive, buffer, process and transmit signals. The detection unit may process the signals and conduct a simplified comparison, e.g., using two factors of amplitude and frequency, with the generalized seizure detection requirements stored in the detection unit. When the detection unit determines that a seizure is occurring, it can download both its analysis and the raw signal data to a bedside base station for more complex processing. The base station may have much more power, larger storage capability and greater processing speed and power, and be better able overall to process the information. It could have a larger database of patterns to compare against. As the seizure detection system “learns” the patient's patterns, the base station may modify the generalized seizure detection requirements to more closely model the patient's pattern. The base station may update the detection device periodically with the modified generalized seizure detection requirements. Likewise, the base station may transmit raw and processed signal data to a remote computer for further analysis and aggregation with signal data from other units in use. For example, multiple base stations may transmit data for multiple patients to a remote computer. Each base station may not receive the other base station's data, but the remote computer may serve as a common repository for data. Aggregation of the data may allow further data points upon which to further refine the generalized seizure detection requirements, thresholds and statistical information that may be supplied to base stations and detection units as a factory default.

In some embodiments, in addition to using EMG and audio electrocardiography (ECG) may be used to corroborate (or contradict) the occurrence of a seizure. This option could be used with particularly difficult patients. Patients with an excessive amount of loose skin or high concentrations of adipose tissue may be particularly difficult to monitor. For example, a factor associated with reliable EMG measurements, is the stability of the contact between the electrodes and skin. For some patients this may be difficult to achieve in a reliable manner. ECG data may be included in a method for determining a likelihood of whether a seizure related incident is taking place (or has taken place) and ECG data may be used to determine whether a seizure should be declared, e.g., an alarm initiated. Moreover, skin and fat are inherently a type of frequency filter. Display of information associated with any devices in addition to EMG and audio, if used, may, in some embodiments, be achieved by clicking or selecting a suitably labeled tab. An authorized user may, for example, be able to open a window that includes access to other data used to corroborate (or contradict) the occurrence of a seizure.

Heart rate may, for example, elevate during a seizure, e.g., a patient may become tachycardic. As discussed further herein, if the EMG processing portion of the seizure detection apparatus determines that a seizure may be in progress and the heart rate does not go up, then the confidence of the detection may be reduced. For example, epileptic patients that use a beta blocker drug may not experience a rise in heart rate, in such situations, a method incorporating heart rate as a factor may be provided with a coefficient to lower the weight given to that factor. Thus, the disclosed detection method and apparatus may be adjusted or readily customized according to patient-specific considerations, such as use of a particular drug regimen. In some embodiments, ECG may be used to detect other cardiac dysrhythmia, such as bradycardia or asystole following a seizure, and to send an alarm if such a condition is detected. Data from a temperature sensor situated as to detect patient temperature may also be used to corroborate occurrence of a seizure or to initiate an alarm.

Generally, the devices of a seizure detection system may be of any suitable type and configuration to accomplish one or more of the methods and goals disclosed herein. For example, a server may comprise one or more computers or programs that respond to commands or requests from one or more other computers or programs, or clients. The client devices may comprise one or more computers or programs that issue commands or requests for service provided by one or more other computers or programs, or servers. The various devices in FIG. 1, e.g., 12, 13, 14, 16, 17, 18 and/or 19, may be servers or clients depending on their function and configuration. Servers and/or clients may variously be or reside on, for example, mainframe computers, desktop computers, PDAs, Smartphones (such as Apple's iPhone™, Motorola's Atrix™ 4G, Motorola's Droid™, Samsung's Galaxy S™, Samsung's Galaxy Note™, and Research In Motion's Blackberry™ devices), tablets (such as Sony's Xperia™, Samsung's Galaxy Tab™, and Amazon Kindle™) netbooks, portable computers, portable media players with network communication capabilities (such as Microsoft's Zune HD™ and Apple's iPod Touch™ devices), cameras with network communication capabilities, smartwatches, wearable computers, and the like.

A computer may be any device capable of accepting input, processing the input according to a program, and producing output. A computer may comprise, for example, a processor, memory and network connection capability. Computers may be of a variety of classes, such as supercomputers, mainframes, workstations, microcomputers, PDAs and smartphones, according to the computer's size, speed, cost and abilities. Computers may be stationary or portable, and may be programmed for a variety of functions, such as cellular telephony, media recordation and playback, data transfer, web browsing, data processing, data query, process automation, video conferencing, artificial intelligence, and much more.

A program may comprise any sequence of instructions, such as an algorithm, whether in a form that can be executed by a computer (object code), in a form that cart be read by humans (source code), or otherwise. A program may comprise or call one or more data structures and variables. A program may be embodied in hardware or software, or a combination thereof. A program may be created using any suitable programming language, such as C, C++, Java, Perl, PHP, Ruby, SQL, and others. Computer software may comprise one or more programs and related data. Examples of computer software include system software (such as operating system software, device drivers and utilities), middleware (such as web servers, data access software and enterprise messaging software), application software (such as databases, video games and media players), firmware (such as device specific software installed on calculators,keyboards and mobile phones), and programming tools (such as debuggers, compilers and text editors).

Memory may comprise any computer-readable medium in which information can be temporarily or permanently stored and retrieved. Examples of memory include various types of RAM and ROM, such as SRAM, DRAM, Z-RAM, flash, optical disks, magnetic tape, punch cards, EEPROM. Memory may be virtualized, and may be provided in, or across one or more devices and/or geographic locations, such as RAID technology. An I/O device may comprise any hardware that can be used to provide information to and/or receive information from a computer. Exemplary I/O devices include disk drives, keyboards, video display screens, mouse pointers, printers, card readers, scanners (such as barcode, fingerprint, iris, QR code, and other types of scanners), RFID devices, tape drives, touch screens, cameras, movement sensors, network cards, storage devices, microphones, audio speakers, styli and transducers, and associated interfaces and drivers.

A network may comprise a cellular network, the Internet, intranet, local area network (LAN), wide area network (WAN), Metropolitan Area Network (MAN), other types of area networks, cable television network, satellite network, telephone network, public networks, private networks, wired or wireless networks, virtual, switched, routed, fully connected, and any combination and subnetwork thereof. The network may use a variety of network devices, such as routers, bridges, switches, hubs, repeaters, converters, receivers, proxies, firewalls, translators and the like. Network connections may be wired or wireless, and may use multiplexers, network interface cards, modems, IDSN terminal adapters, line drivers, and the like. The network may comprise any suitable topology, such as point-to-point, bus, star, tree, mesh, ring and any combination or hybrid thereof.

Wireless technology may take many forms such as person-to-person wireless, person-to stationary receiving device, person-to-a-remote alerting device using one or more of the available wireless technology such as ISM band devices, WiFi, Bluetooth, cell phone SMS, cellular (CDMA2000, WCDMA, etc.), WiMAX, WLAN, and the like.

Communication in and among computers, I/O devices and network devices may be accomplished using a variety of protocols. Protocols may include, for example, signaling, error detection and correction, data formatting and address mapping. For example, protocols may be provided according to the seven-layer Open Systems Interconnection model (OSI model), or the TCP/IP model.

Although the foregoing details describe certain embodiments of this invention, persons reasonably skilled in the art will recognize that various changes may be made in the details of this invention without departing from the spirit and scope of the invention as defined in the appended claims and considering the doctrine of equivalents. Therefore, it should be understood that this invention is not to be limited to the specific details shown and described herein.

Additional information related to the methods and apparatus herein described may be understood in connection with the examples provided below.

### EXAMPLE 1

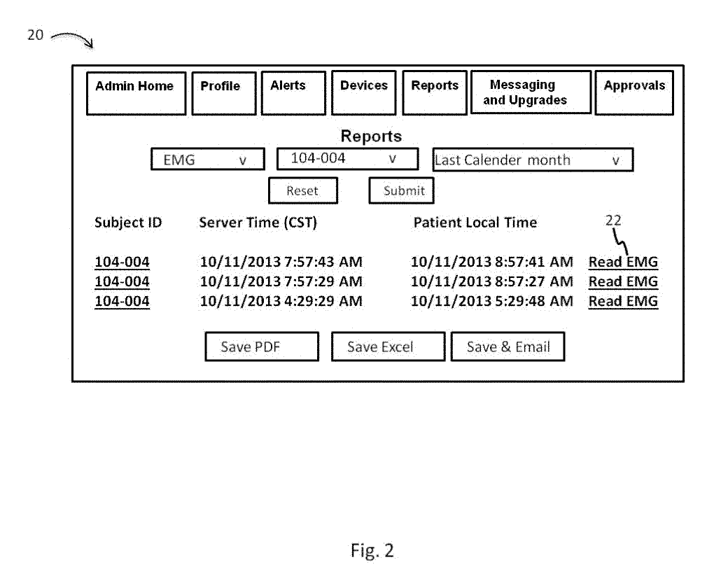

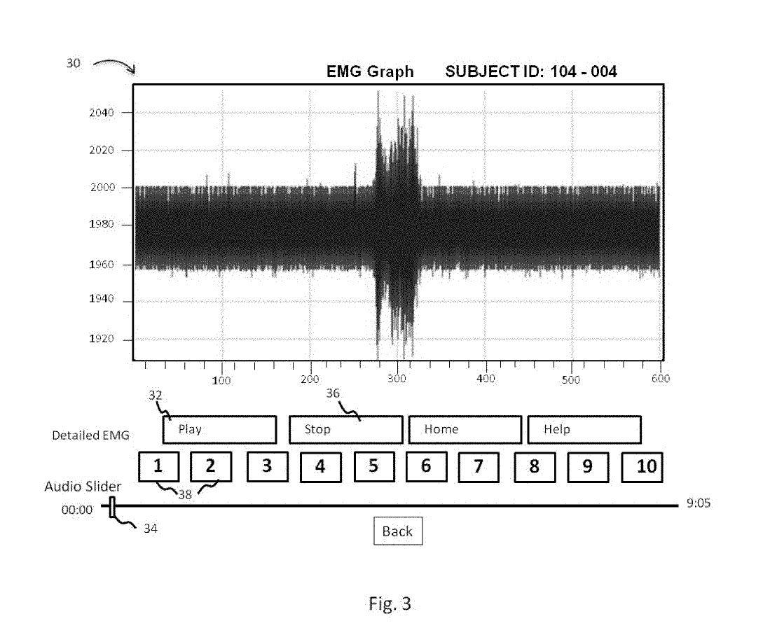

In this Example 1, various patients were monitored using a combination of both EMG and audio sensors. For any patient who had a detected seizure, the system was configured to automatically send a recording of the EMG and audio signal to a remote server for access and analysis by physicians or other authorized people. Transmission of data may be executed in a secure manner using HIPAA compliant security measures. Data may be sent using an identifier that may be correlated with a specific patient identity after correlation using a remote database. For the system in this example, a 5 minute period prior to a detected event and a 5 minute period following a detected event is automatically transmitted upon detection of an event that may be a seizure. The EMG and audio data are synchronized so that not only can the EMG signal characteristics be analyzed, but sounds emitted by the person who may be having a seizure are aligned with the event. During the period of analysis in this example, 3 separate recorded events were identified for one of the subject patients. An authorized person may be able to view a listing of recorded events. FIG. 2 shows a screen shot 20 listing of 3 events marked by a patient identifier and identified by an event time. Selection of an appropriate tab or button may then be used to open up a graph with buttons allowing viewing of the entire 10 minutes of EMG data. For example, by clicking on the “Read EMG” button 22, an authorized user may open up a window showing EMG data and allowing access to recording sound data as further described in FIG. 3 of Example 2.

### EXAMPLE 2

In this Example 2, as shown in FIG. 3, a screen shot 30 of EMG data for a selected patient and event is displayed. By selection of the “Play” button 32, an authorized user can listen to all of or a part of the recorded audio data. A user may further drag an audio slider 34 to any part of the graph and listen to recorded sound synchronized with the EMG data. A “Stop” button 36 may be used to stop the playing of recorded sound and to freeze the slider at any desired time within the recorded data. A user may further select any of the “Detailed EMG” buttons 38 to zoom in on a particular time period of the data. For example, clicking on the “Detailed EMG” button labeled one will display the first minute of recorded data.

### EXAMPLE 3

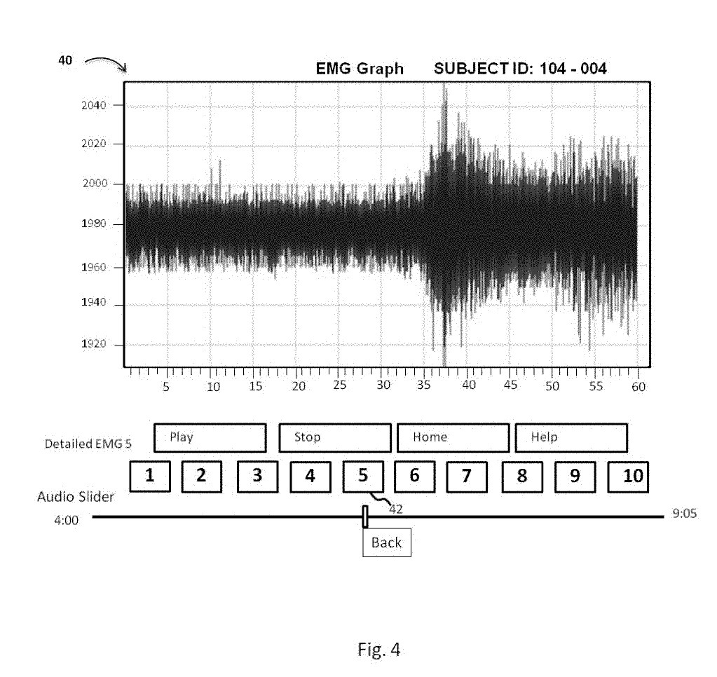

In this Example 3, as shown in FIG. 4, a screen shot 40 of EMG data for a one minute period of data is shown as may be displayed upon selection of one of the “Detailed EMG” buttons. In the screen shot 30 the 5 minute mark button 42 has been selected. Upon selection of the button 42 a one minute period of data is displayed. The selected button may further be shown as highlighted or identified with another convenient marking so that a user may readily verify the specific period of data displayed.

### EXAMPLE 4

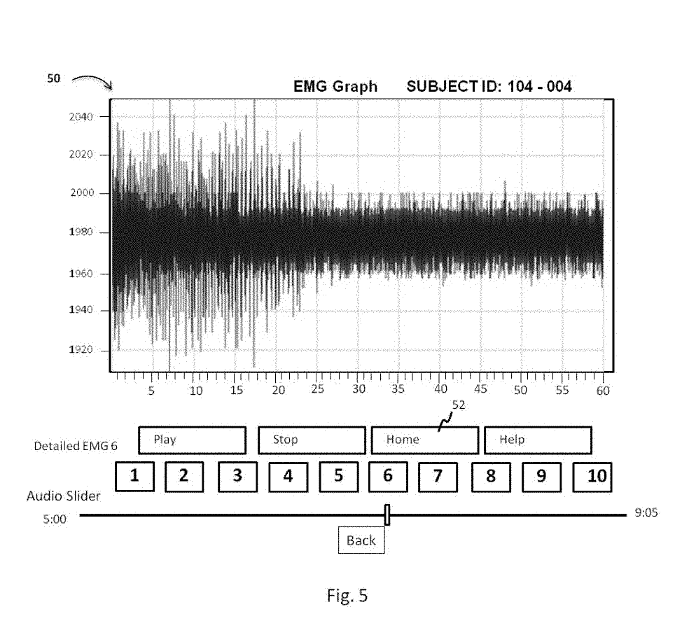

In this Example 4, as shown in FIG. 5, another screen shot 50 of EMG data for another one minute period of data is shown. By clicking on the “Home” button 52 a user may move from a zoomed in view to an expanded view, e.g., all 10 minutes of recorded data in this Example. The audio data will automatically synchronize with the selected time period and can be played.

### EXAMPLE 5

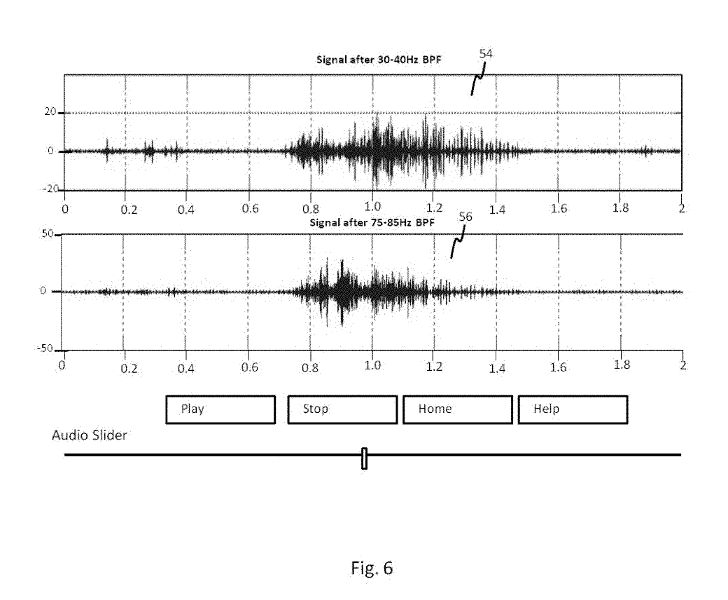

In this Example 5, as shown in FIG. 6, a split-screen view showing two different spectral regions of EMG data (54, 56) may be displayed. In FIG. 6, a two minute block of data is displayed for seizure data within a first band including spectral data from about 30-40 Hz (54) and a second band from about 75-85 Hz (56).

Although the disclosed method and apparatus and their advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition, or matter, means, methods and steps described in the specification. Use of the word “include,” for example, should be interpreted as the word “comprising” would be, i.e., as open-ended. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods or steps.

1. A method of reviewing data collected for a patient susceptible to seizure activity comprising:
monitoring a patient using an EMG electrode and an acoustic sensor so as to obtain first EMG data and first audio data;
determining whether the first EMG data and optionally the first audio data exceeds a threshold value;
if said threshold value is exceeded, then sending the first EMG data and the first audio data to a remote server and storing the first EMG data and the first data on the remove server;
for accessing and processing the stored first EMG data and the stored first audio data on said remote server;
wherein the processing of said EMG data and said audio data includes displaying the first EMG data on a graphical user interface and enabling a user to play back the first audio data through the graphical user interface synchronously with the first EMG data; and
providing a marker on the graphical user display as associated with the first EMG data and the played first audio data.

2. The method of claim 1 wherein the marker is an audio slider located on or beneath an axis of the graphically-displayed first EMG data.

3. The method of claim 1 further comprising storing second EMG data and second acoustic data in a buffer and if said threshold value is achieved, then sending the buffered second EMG data and second acoustic data along with the first EMG and first audio data.

4. The method of claim 3 further comprising if said threshold value is not met within a period of about 2 to about 8 minutes, then deleting the second EMG data and second acoustic from said buffer.

5. The method of claim 4 further comprising if said threshold value is not met within a period of about 5 minutes, then deleting the second EMG data and second acoustic from said buffer.

6. The method of claim 1 wherein said threshold value for the first acoustic data ranges between about 50 decibels to about 75 decibels.

7. The method of claim 1 wherein the processing of said first EMG data and said first audio data further includes graphing a characteristic of the first audio data detected by said acoustic sensor;
wherein said characteristic is selected from the group of characteristics consisting of amplitude, amplitude at a given frequency or over a certain frequency range, rate of change of amplitude, spectral slope, periodicity, and combinations thereof.

8. A computer programmed with a computer program for:
receiving audio and EMG data from a remote server;
displaying said EMG data and enabling selection of different portions of the displayed EMG data; and
playing audio data that is synchronized with said selected EMG data.

9. The computer of claim 8 further programmed for:
displaying a characteristic of said audio data;
wherein said characteristic is selected from the group of characteristics consisting of
amplitude, amplitude at a given frequency or over a certain frequency range, rate of change of amplitude, spectral slope, periodicity, and combinations thereof.

10. The computer of claim 8 wherein the displayed characteristic of said audio data is provided as a split screen along with the displayed EMG data.

11. The computer of claim 8 wherein the displayed EMG data includes an amplitude of EMG data for all monitored frequencies of said EMG data.

12. The computer of claim 8 wherein the displayed EMG data includes an amplitude of EMG data for spectral data derived from one or more frequency bands included among the EMG data.

13. The computer of claim 8 wherein the displayed EMG data is a split-screen that includes an amplitude of EMG data for spectral data derived from one frequency band and an amplitude of EMG data for spectral data derived from another frequency band.

14. A computer readable medium comprising instructions for:
receiving audio and EMG data from a remote server;
displaying said EMG data and enabling selection of different portions of the displayed EMG data; and
playing audio data that is synchronized with said selected EMG data.

15. The computer readable medium of claim 14 further comprising instructions for:
displaying a characteristic of said audio data;
wherein said characteristic is selected from the group of characteristics consisting of amplitude, amplitude at a given frequency or over a certain frequency range, rate of change of amplitude, spectral slope, periodicity, and combinations thereof.

16. The computer readable medium of claim 14 wherein the displayed characteristic of said audio data is provided as a split screen along with the displayed EMG data.

17. The computer readable medium of claim 14 wherein the displayed EMG data includes an amplitude of EMG data for all monitored frequencies of said EMG data.

18. The computer readable medium of claim 14 wherein the displayed EMG data includes an amplitude of EMG data for spectral data derived from one or more frequency bands included among the EMG data.

19. The computer readable medium of claim 14 wherein the displayed EMG data is a split-screen that includes an amplitude of EMG data for spectral data derived from one frequency band and an amplitude of EMG data for spectral data derived from another frequency band.
